(19) United States
(12) Patent Application Publication
(10) Pub. No.: US 2007/0212719 A1
(43) Pub. Date: Sep. 13, 2007

(54) GRAPHICAL RULE BASED MODELING OF BIOCHEMICAL NETWORKS

(75) Inventors: William S. Hlavacek, Santa Fe, NM (US); James R. Faeder, Santa Fe, NM (US); Michael L. Blinov, Avon, CT (US)

Correspondence Address: JOHN P. O'BANION;O'BANION & RITCHEY LLP, 400 CAPITOL MALL SUITE 1550, SACRAMENTO, CA, 95814, US

(73) Assignee: LOS ALAMOS NATIONAL SECURITY, Los Alamos, NM (US)

(21) Appl. No.: 11/685,114

(22) Filed: Mar. 12, 2007

Related U.S. Application Data
(60) Provisional application No. 60/781,571, filed on Mar. 10, 2006.

(51) Int. Cl. C12Q 1/68; G06F 19/00

(52) U.S. Cl. 435/6; 702/20

(57) ABSTRACT

Formalized graphical reaction rules and conventions accounting for chemical states and binding or reaction states are provided for modeling complex biological systems such as signal transduction pathways. A system model is derived by defining typed attributed graphs which delimit molecular entities and their possible states. Graph transformation rules defining a class of potential reactions are defined and applied to the graphs and all new graphs that subsequently arise as a result of graph transformation. In one embodiment, a model is generated through the use of graph-rewriting rules which are associated with rate laws and applied iteratively to a seed set of chemical species graphs until a termination condition occurs.

### CROSS-REFERENCE TO RELATED APPLICATIONS

This application claims priority from U.S. provisional application Ser. No. 60/781,571 filed on Mar. 10, 2006, incorporated herein by reference in its entirety.

### STATEMENT REGARDING FEDERALLY SPONSORED RESEARCH OR DEVELOPMENT

This invention was made with Government support under Grant No. GM35556 and RR18754 awarded by The National Institute of Health as well as Department of Energy Contract No. W-7405-ENG-36. The Government has certain rights in this invention.

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention pertains generally to complex network modeling, and more particularly to graph based computational methods for modeling biochemical networks and cellular systems.

2. Description of Related Art

Biological systems typically involve cascades of chemical reactions, intermolecular associations and cellular signaling. For example, signal transduction, one process by which a cell senses and responds to its environment, is often mediated by a network of protein-protein interactions, in which proteins combine to form complexes and undergo post-translational modifications, which regulate their enzymatic and binding activities. A typical signaling protein contains multiple sites of protein interaction and modification and may contain catalytic domains. Many cellular responses to environmental signals that are mediated by networks of interacting proteins detect signals (e.g., ligands of cell-surface receptors) and transduce these signals into cellular responses, such as the release of stored factors, changes in gene expression, and cell movement, proliferation, differentiation, or death.

After the introduction of a signal, many proteins in a signal-transduction network may undergo post-translational modifications (e.g., tyrosine phosphorylation), which affect their binding and enzymatic activities, and concurrently combine to form a variety of heterogeneous complexes. These complexes, which are often transient and prominent in the vicinity of the inner cell membrane, regulate enzymatic activities, for example, by serving to co-localize enzymes and substrates, which is a common mechanism for controlling enzyme specificity. The number of protein complexes and modification states that potentially can be generated during the response to a signal is combinatorially large and generally far greater than the number of proteins involved in signal transduction, because signaling proteins contain multiple sites of modification and may interact with multiple binding partners.

Accordingly, a typical signaling protein contains multiple sites that may be reversibly modified (e.g., phosphorylated), bind other proteins or lipids, or regulate the protein's enzymatic activity, giving rise to many different protein states and therefore a large number of potential signaling molecular complexes. For example, a protein that contains 10 amino acid residues (N=10) subject to the activities of kinases and phosphatases theoretically has 2¹⁰=1024 states of phosphorylation. Therefore, there is a need to account for 2^{N} or 1024 multi-state species in this example. If dimerization is required for signaling, one has to consider on the order of 2^{2N}=1,048,576 receptor dimers i.e. multi-component, multi-state species. Since each phosphorylation site may bind proteins that can be in different states themselves, the number of different states and possible molecular species goes up dramatically. Consequently, representing signal-transduction networks can be a serious challenge.

Because the full spectrum of protein states and complexes is difficult to enumerate, let alone understand, it is likely that mathematical/computational modeling will need to play an important role in interpreting such data and assessing the functional significance of specific interactions and complexes. Unfortunately, combinatorial complexity has been largely ignored by both experimentalists and modelers and is a major barrier to predictive understanding of signal transduction and other complex biological systems.

Experimental resolution of protein states and complexes has been limited to a small number of sites and interactions, but rapidly advancing proteomic technologies are likely to provide a wealth of more detailed information about signaling complexes in the future. Mathematical modeling can be a valuable interpretive and predictive tool for understanding cell signaling cascades and other inter-cellular and intra-cellular systems. However, current models are limited and do not encompass the breadth of chemical species required to fully describe complex networks such as those found in biological systems. Instead, most models, given a particular set of proteins and interactions, are based on assumptions (usually implicit) that exclude the vast majority of possible molecular species from consideration.

Accordingly, there is a need for a model creation method that accounts for all of the possible protein states and molecular complexes that are present in a signal transduction network or other biological system.

There is a need for a modeling system that can help catalog potential molecular species involved in signaling or other biological activities and predict the molecular species that are actually involved in the pathways and determine the functional importance of the processes involved in the creation of the molecular species.

There is a further need to provide a modeling method that will provide mathematical an computational models that can be used to interpret acquired data, predict the behavior of a system, and permit the design of experiments to test the model-based predictions.

The present invention satisfies these needs as well as others and generally overcomes the deficiencies of the art.

### BRIEF SUMMARY OF THE INVENTION

Many cellular responses to environmental signals are mediated by networks of interacting proteins that detect signals (e.g., ligands of cell-surface receptors) and transduce these signals into responses, such as the release of stored factors, changes in gene expression, and cell movement, proliferation, differentiation, or death. After the introduction of a signal, the proteins in a signal-transduction network typically undergo post-translational modifications (e.g., tyrosine phosphorylation), which affect their binding and enzymatic activities, and concurrently combine to form a variety of heterogeneous complexes. These complexes, which are often transient and prominent in the vicinity of the inner cell membrane, regulate enzymatic activities, for example, by serving to co-localize enzymes and substrates, which is a common mechanism for controlling enzyme specificity.

A common feature of biochemical networks, especially those comprising protein-protein interactions, is combinatorial complexity, which is present whenever a small number of biomolecular interactions have the potential of generating a much larger number of chemical species and reactions. Combinatorial complexity must be addressed in signal transduction network modeling at the molecular level. For example, interactions between proteins are mediated by sub-molecular components, such as protein motif or protein interaction domains, and most proteins have multiple interactive sites and components.

Additionally, protein interactions depend on molecular context, which often influences the enzymatic or binding activities of the protein. For example, the rate of an enzymatic reaction may depend on the co-localization of an enzyme and its substrate, and the binding of the two interacting proteins may depend on the phosphorylation state of one or both of these proteins.

Accordingly, tracking protein-protein interactions generally requires an accounting of multivalent interactions and molecule or component states. A mechanistic model should account for all possible multicomponent complexes and all possible states of molecules in a system. There are at least two reasons to account for all the possible protein states and complexes in a signal-transduction networks and networks with molecular interactions. First, it is impossible to determine intuitively which states and complexes are functionally important from knowledge of pairwise protein interactions, which is the usual level of detail available, even for a well-studied system. Secondly, the catalytic activities of signaling proteins are highly regulated by molecular context. For example, the activity of a protein tyrosine kinase (PTK) might depend on the phosphorylation state of its activation loop and its specificity might depend on the proximity of a specific substrate.

By way of example, and not of limitation, a preferred method for the modeling of biochemical networks using graphical, rule based conventions is generally described that accounts for all chemical species, modified states, complexes, component interactions, reactions and other relevant conditions. The graph-theoretic formalism of the method is tailored to the problem of building physicochemical models of biochemical networks, particularly protein-protein interaction networks. It allows for the abstraction of proteins, functional components of proteins, and protein complexes, including multimeric proteins that function as a unit. Although a signal transduction pathway is used to illustrate the method, it will be understood that the method can be applied to other types of cellular systems and biomolecular interactions, such as genetic regulatory networks and protein-DNA or protein-lipid interactions and the like.

The methods of the present invention use graphs and graph rewriting rules, or graphical reaction rules, to represent biochemical networks. The graphical representation of networks precisely accounts for the full array of possible protein states and complexes implied by a given set of protein interactions. Mathematical and computational models are created from the graphical rule based representations, which can then be used to interpret data, predict the behavior of a system, and design experiments to test model-based predictions.

Typed attributed graphs are used to represent proteins and their functional components, complexes, conformations, and states of post-translational covalent modification and other characteristics relevant to the system. In the embodiment shown, the elements of a graph are nodes, labels associated with the nodes, and undirected and unlabeled edges that connect nodes. Labeled nodes represent components of proteins and their states, and edges represent bonds between components.

Graph transformation rules are used to represent biomolecular interactions and their effects. Each rule defines a generalized reaction or a class of potential reactions (e.g. binding or enzymatic reactions) that are consistent with the knowledge or assumptions about the represented biomolecular interaction.

According to one embodiment, a model may be created by defining (1) molecular entity graphs, which delimit the molecular entities and their possible states, (2) graph transformation rules, and (3) a seed set of graphs representing chemical species, such as the initial species present before the introduction of a signal. The iterative application of the graph transformation rules provides a reaction network. The rules are first applied to the seed graphs and then to any and all of the new graphs that subsequently arise as a result of graph transformation until no new graphs are produced or a termination condition is satisfied.

According to one aspect of the invention, a method for generating a list of reactions among chemical species in a system is provided by using graphs to represent material components of the system and their physical connections, including molecules, complexes of molecules, parts of molecules, states of molecules, states of parts of molecules, and chemical bonds and the like. Graph-rewriting rules, comprising graphs that represent molecular features of reactants and products and a mapping of molecules and molecular components from reactants to products, are created to represent the creation of bonds, destruction of bonds, changes of states of molecules, changes of states of molecular components, synthesis of molecules, degradation of molecules, and transport of molecules between compartments etc. The graph-rewriting rules may be applied using a well-known single-pushout approach to transform graphs and generate connections between graphs representing chemical reactions and transformations.

According to another aspect of the invention, a method is provided wherein the graphs represent small-molecule metabolites, the vertices represent atoms of these metabolites (e.g., the subset of atoms that are carbon atoms), the vertex labels represent the isotopic form of these atoms (i.e., the mass number), the graph-rewriting rules represent enzyme-catalyzed and spontaneous metabolic reactions, and the map included in each rule defines the fates of the atoms in reactions generated by the corresponding rule.

In another aspect of the invention, a chemical reaction network may be obtained that defines the isotopomers that can be generated by isotopic labeling of at least one metabolite that is a member of the metabolic network defined by the list of rules.

According to another aspect of the invention, graphs and graph rewriting rules that are encoded in machine readable format are archived in an electronic database or an association of databases facilitating the generation of reaction networks.

A further aspect of the invention is to provide a simulation procedure that accurately predicts the dynamics of a biological network by converting, for example, the network into a coupled system of ordinary differential equations and numerical integration of the equations.

In yet another aspect of the invention, computer hardware and software are provided that is configured to generate graphs and graph rewriting rules and visually represent a reaction network resulting from the application of the rules as well as simulate changes in the network with changes in the input graphs, rules or reaction rates etc.

Accordingly, it is an object of the present invention to provide a method for generating reaction networks of biomolecular interactions.

It is yet another object of the present invention to provide a method for generating a reaction network that considers a significantly larger list of possible chemical species and interactions among them than previous methods.

A further object of the invention is to provide a method that accounts for all possible protein states and complexes in a signal transduction network implied by a given set of protein interactions.

Additional objects, advantages, aspects and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims and specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a flowchart of one embodiment of a method for graphical rule based modeling of biochemical networks.

FIG. 2 is a list of illustrative graphical representations of conventions of the method for graphical rule based modeling of biochemical networks according to the present invention.

FIG. 3A and FIG. 3B is a flowchart of another embodiment of the method for generating a model of a complex chemical reaction network through the iterative application of graph transformation/reaction rules on an ensemble of chemical species graphs according to the present invention.

FIG. 4A-E depicts different types of reactions illustrating rewriting operations according to the present invention.

FIG. 5A is a chart of molecular definitions for the FcεRI network model according to the present invention.

FIG. 5B is a representative chemical species graph for the FcεRI network described in Example 1.

FIG. 5C is a representative component level type graph for the FcεRI network described in Example 1.

FIG. 5D is a representative chemical species graph of FIG. 4B typed over the component level type graph of FIG. 4C for the FcεRI network described in Example 1.

FIG. 5E is a representative pattern graph for the FcεRI network described in Example 1.

FIG. 5F is a representative ensemble of chemical species graphs matched by the pattern graph of FIG. 4E for the FcεRI network described in Example 1.

FIG. 6 is a list of the reaction rules for graphical rule based representation of the schematic of the FcεRI network described in Example 1.

FIG. 7A is a reaction rule for ligand receptor binding with a reactant pattern graph (RP) and a product pattern graph (PP) mapping f.

FIG. 7B is a reaction that may be generated by the reaction rule of FIG. 6A.

FIG. 8 is a prior art map of the phosphorylation of the retinoblastoma protein (Rb) by a cyclin-dependent kinase (cdk).

FIG. 9 is a chart of the molecular definitions and reaction rules for the map of FIG. 8 according to the invention.

### DETAILED DESCRIPTION OF THE INVENTION

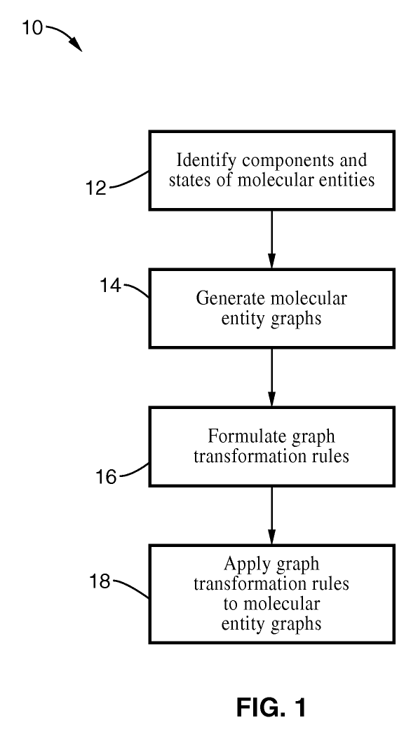

Referring more specifically to the drawings, for illustrative purposes the present invention is embodied in the methods generally shown in FIG. 1 through FIG. 9. It will be appreciated that the methods may vary as to configuration and as to details of the parts, or may vary as to the specific steps and sequence, without departing from the basic concepts as disclosed herein.

Referring first to FIG. 1, a method 10 for creating a biochemical reaction network for interacting biomolecules is provided that accounts for all biomolecules, bimolecular complexes, internal states and other chemical species, that are implied by specified interactions among the biomolecules. Molecular entity graphs 14 are used to define material parts of the system.

The graphical conventions of the method considers the components of biomolecules, such as binding domains, sites of enzyme-controlled modification, and domains of catalytic activity, as well as internal states. Graph transformation rules 16 are applied 18 to generate a biochemical reaction network.

A computer device and software may be used to implement the method and generate biochemical reaction networks of the invention for a wide array of cellular systems.

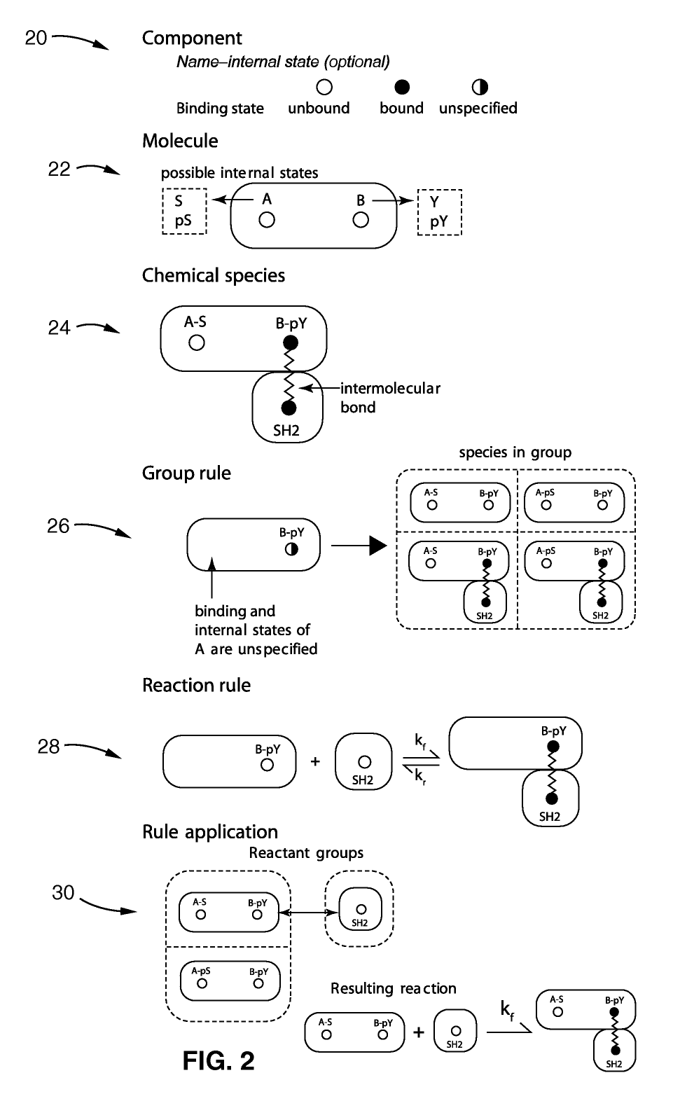

Components and states of the molecular entities of a selected network are identified and defined at block 12 of method 10 shown in FIG. 1. Referring also to FIG. 2, components 20 of the molecular entities of a network are inherent to the molecule and may be identified by their function or structure. Proteins involved in signal transduction, for example, typically contain multiple functional components and interactions are mediated by such components. Examples include sites of modification (amino acid residues), protein motifs, catalytic subunits, and protein interaction domains.

Some domains are responsible for biomolecular recognition; protein interaction domains recognize specific types of sites in proteins and other biomolecules. For example, the Src homology 2 (SH2) domain recognizes phosphorylated tyrosines in protein motifs, such as the immunoreceptor tyrosine-based activation motif (ITAM). The activities of protein domains can be regulated by post-translational modifications, which are catalyzed by signaling proteins. These modifications can also be reversed (e.g., a tyrosine can be dephosphorylated by a protein tyrosine phosphatase).

Binding events and conformational changes can also affect the activities of signaling proteins. The methods account for the interactions among molecules that may have multiple components with binding or catalytic activity that is dependent on bound, conformational, or modification state, which can vary.

Accordingly, phosphorylation status is one example of an internal state of a component or a molecule. The two possible states of such a protein component might be labeled ‘phosphorylated’ and ‘not phosphorylated.’

Another example of internal status is the three-dimensional conformation of a protein. If consideration of two conformations is adequate for modeling purposes, these states might be labeled ‘open’ and ‘closed.’ A specification of a molecular entity may also include a definition of the chemical transformations that can potentially take place in a system. For example, some transformations may change the connectivity of molecular parts such as when two proteins form a complex. Other transformations may change the internal states of molecular parts, as when a protein tyrosine kinase (PTK) catalyzes a phosphorylation reaction or when binding of a ligand induces a conformational change of an allosteric enzyme.

Molecular entities may also be comprised of a complex of molecular entities, each entity in the complex having its own set of components and states.

Molecular entity graphs are generated at block 14 of FIG. 1. The preferred elements of an entity graph are nodes, labels associated with the nodes, and undirected and unlabeled edges that connect nodes. Nodes represent components (e.g., sites and domains of proteins), which may have multiple internal states (e.g., phosphorylated or unphosphorylated), labels give the names of components and their internal states, and edges represent bonds between components. Note that edge labels can be introduced (e.g., to distinguish between different types of bonds) and edges can be directed (e.g., to indicate parent-child relationships among the components of a molecular entity).

It is preferred that represented edges are subject to addition or removal in a graph rewriting step, i.e., bonds affected by signaling or other system function. Bonds connecting components that are unaffected by signaling are not represented explicitly in this embodiment.

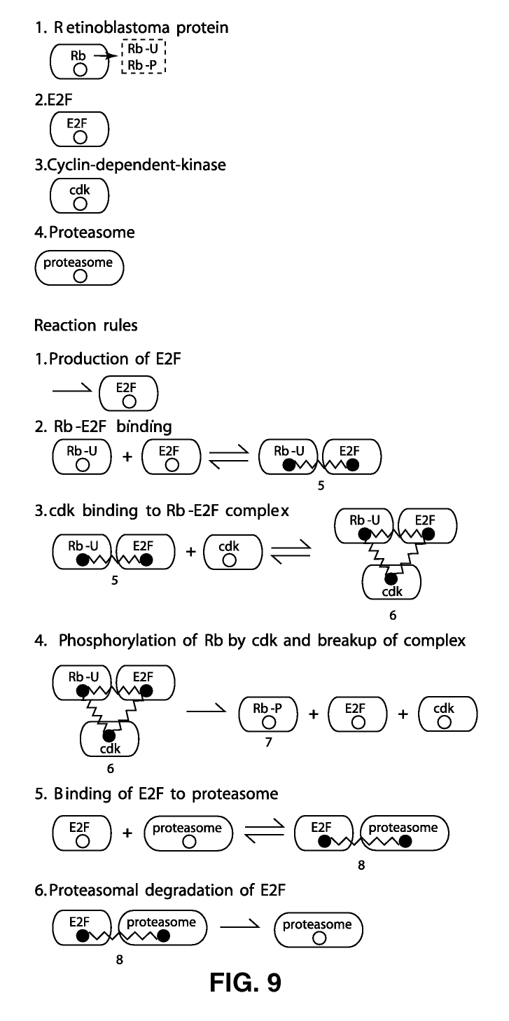

Internal states are introduced as needed or desired to represent bound, conformational, or modification states of a component that are not represented otherwise. As illustrated in FIG. 2 and 9, when a component 20 is defined, it is assigned a name and a list of its allowed internal states (if any) is given.

In some settings it will be necessary to specify the connectivity of a node, for example, to write a reaction rule in which a particular component of a reactant must be unbound. In the embodiment shown in FIG. 2, an open (filled) circle is used to denote a node that is unconnected (or connected) to an edge. A half-filled circle is used for a node that may be either connected or unconnected to an edge. Other ways of specifying connectivity are possible. For example, a special node might be introduced to represent an empty space and connected to nodes of components that are unbound.

A molecule 22 is defined as a set of components that can be treated as a unit, such as the components of a polypeptide chain or multimeric protein. As shown in FIG. 2, a molecule 22 is represented graphically by a box surrounding a set of nodes that represents each component of the molecule. Like components 20, molecules 22 may be assigned names. However, in one embodiment, names are suppressed to avoid clutter, because molecules 22 can be distinguished by the shapes of their boxes or the names of their components. Names of components 20 may also be suppressed in some cases.

In one embodiment, names can be suppressed because of the convention that the components of a molecule are represented at fixed relative positions within a box. These conventions for illustrating a model do not affect the underlying graph representation of components, bonds, and molecules. The internal states of a molecule and its connectivity to external components are determined by the attributes of the nodes representing its components.

A chemical species 24 of FIG. 2, is generally either a single molecule having all of its components fully defined or a set of connected molecules (i.e., a complex), with each molecule in the set having all of its components fully defined. A component 20 is fully defined if its internal state is specified and its connection with other components is specified. If a component is bound to another component, then the nodes representing the two components are joined by an edge.

In general, a chemical species 24 is represented by a graph in which nodes are partitioned into molecules, edges connect the nodes of components that are bound to each other, and node labels indicate the particular internal states of those nodes that have multiple allowed states. There is a chemical species for each unique combination of the possible component connections and states in a system.

In one embodiment, edge labels are provided to distinguish between edges that can and cannot be added or removed through graph rewriting. Edge labeling provides a representation of the internal connectivity of the components in a molecule. In another embodiment, ternary or higher order interactions between components are accounted for through the use of valence edge labels.

Groups of chemical species 26 with specified shared features can be defined by graphs that do not completely specify component interactions and states, which are called group rules or group graphs. The interactions and states that are specified define the distinguishing features of a group. An example of a group rule 26 is shown in FIG. 2, along with a set of chemical species having features consistent with the rule. Generally, given a set of chemical species, this group rule selects all chemical species among the set in which component B of the indicated molecule is in state pY as shown in the example.

Because the internal state of component A and the connectivity of B are unspecified in the group rule 26, chemical species selected by the rule can have different states of A and different bound states of B as shown in FIG. 2. Additional species, depending on the set of species being tested, could belong to the group, as would be the case if component A was attached to a binding partner in one of the species among the set tested. For example, a chemical species represented by graph X is a member of a group represented by group graph G, if and only if there is a sub graph of X that is isomorphic to G when the internal states are removed from the labels of nodes in X that have unspecified internal states in G. Thus, the problem of identifying which species belong to a group is reduced to the problem of determining whether X contains subgraphs isomorphic to the group graph G.

At block 16 of FIG. 1, the graph transformation rules are generated that represent chemical reactions or other protein-protein interactions and their effects. Each rule defines a generalized reaction i.e. a class of potential reactions that are logically consistent with what is known about the represented biomolecular interaction. For example, graph transformation rules may be graph rewriting rules that can be used to generate chemical reactions from a list of chemical species 24 by identifying sets of reactants and defining how reactants are transformed into products. Each rule 28 is comprised of two sets of group graphs (a set of graphs representing reactants and a set of graphs representing products), an arrow pointing from reactants to products, and a rate law. The rate law in general can be any function of the properties of reactants and products, e.g., k[A][B], where k is a rate constant and [A] and [B] are the concentrations of reactants in a biomolecular reaction. The bidirectional arrows of the reaction rule 28 illustrated in FIG. 2 indicate that the rule is to be applied in both the forward and reverse directions.

It can be seen that there is a close correspondence between the protein interactions in a system and the graph transformation (reaction rules) needed or used to model the system. Importantly, the number of reaction rules needed to represent a system is related to the number of components in the system, which in general is far less than the number of possible chemical species and reactions.

The generated graph transformation rules 16 are applied to the molecular entity graphs at block 18 of FIG. 1. An initial set of chemical species is preferably specified as a starting point for the application of reaction rules 30 and the generation of a chemical reaction network. A typical starting point for network generation would be the set of individual molecules with each component in its resting internal state.

In one embodiment, the first step in applying a reaction rule to a set of chemical species is to identify the group of species corresponding to each reactant group graph 26. Next, for each combination of reactant species drawn from these groups, the reaction rule 28 is applied by replacing the subgraphs of the reactant species matching the group graphs of reactants with the corresponding group graphs of products to define the products. In carrying out this replacement, component states that are not specified in the product group graphs are not changed. This process of replacing subgraphs of reactants with product group graphs is a graph rewriting step (or in some cases, equivalently, a re-labeling operation) that transforms reactant graphs in product graphs.

The product species that result from graph rewriting are then checked against the current list of chemical species and added to the list if they are not already present. To facilitate this comparison, graphs are preferably assigned a unique label that does not depend on the order of components, graph partitions (i.e., molecules), or edges. In one embodiment, the labels are assigned using a canonical graph labeling scheme from among the well known techniques in the art. Canonical labels are also useful for checking the generated reaction against the list of previously generated reactions to identify overlaps in reaction rules or to prevent duplication of reactions that are related because of symmetry.

Iterative application of reaction rules can be carried out until a termination condition is satisfied or all possible species and reactions are generated. An exhaustive generation of all species and reactions accessible from the initial set is a possible termination condition as long as the reaction rules give rise to a finite number of species, but may not be desirable in the case of very large networks, e.g., if the number of chemical species containing a particular molecule exceeds the number of that kind of molecule in a cell. In these cases, other termination conditions are needed. Alternatively, network generation and simulation can proceed in tandem, such that species and reactions are generated on-the-fly as needed.

Additionally, it is often useful to associate a mathematical function with a group of chemical species, such as the sum of concentrations of all members of a group, because experimental observables often correspond to properties of ensembles of chemical species. A group graph and associated function can be specified to calculate this sum. For example, the group rule 26 illustrated in FIG. 2 could be used to calculate the concentration of the specified protein phosphorylated on its B domain tyrosine, and group rules could be used to calculate the concentration of receptor-bound autophos-phorylated Syk.

Accordingly, the graphical rule based approach of the present invention specifies interactions in the form of rules and the complexes and states implied by those rules. It is possible to track connectivity or reactivity of components in molecules or complexes systematically and explicitly and through an automated procedure.

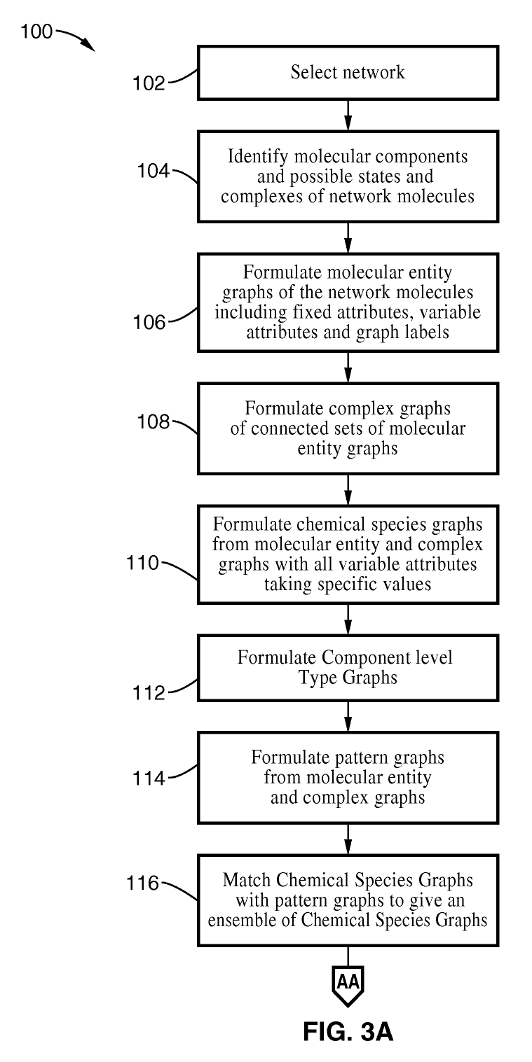

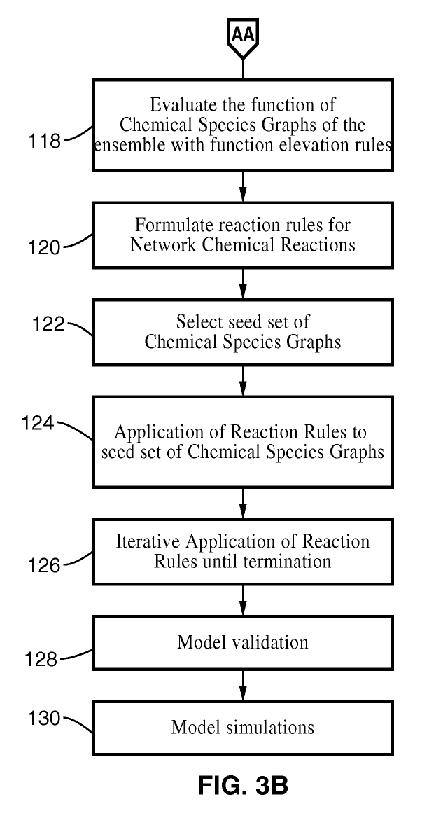

Turning now to FIG. 3A and FIG. 3B, another embodiment of the method 100 for graphical rule based modeling of biochemical networks is shown. Generally, typed attributed graphs are used to represent proteins and their functional components, complexes, conformations and states of post-translational covalent modification and the like in this embodiment. Graph transformation rules are then used to represent protein-protein interactions and their effects. Each rule defines a generalized reaction, i.e. a class of potential reactions that are logically consistent with knowledge and assumptions about the represented biomolecular interaction. Thereafter, a model is specified by defining (1) molecular entity graphs, which delimit the molecular entities and material components of a system and their possible states, (2) graph transformation rules, and (3) a seed set of graphs representing chemical species such as the initial species present before introduction of a signal.

A reaction network model is generated through the iterative application of graph transformation rules. The rules are first applied to the seed graphs and then to any and all new graphs that subsequently arise as a result of graph transformation. This procedure continues until no new graphs are generated or a specified termination condition is satisfied.

Referring now to FIG. 3A, at block 102, a biochemical network for modeling is selected. The method of this embodiment is particularly suited for networks where a small number of biomolecular interactions have the potential to generate a much larger number of distinct chemical species and reactions i.e. having combinatorial complexity. Modeling of such networks can be a valuable interpretive and predictive tool for understanding cell signaling cascades, for example, as well as understanding intercellular associations and system function. Predictive modeling can also direct experimentation and suggest network manipulation or compensation for observed network deficiencies. Thus, models may be generated that are relevant for rational drug discovery, analysis of proteomic data and mechanistic studies of signal transduction.

At block 104, the molecular components and possible states and complexes of the molecules of the selected network are identified. Functional components of interest include sites of modification (amino acid residues), protein motifs, catalytic subunits and protein interaction domains, etc.

Existing network and system information that is available can be collected and utilized at this step to identify the network biomolecules, their composition, known states, known complex formation, functional characteristics and generally define the network landscape. The structure and function of a network protein molecule implies a set of molecular species with combinations of certain states or activity. For example, the typical signaling protein in a signaling network contains multiple domains with multiple sites that can be reversibly modified (e.g. phosphorylated), can bind with other proteins or lipids, or can regulate the enzyme activity of the molecule giving rise to many different possible protein states. It can be seen that the reactivity of domains, potential modifications and interactions can imply a large number of possible molecular species that must be considered to develop predictive models at the molecular level that can asses the functional significance of specific modifications, interactions, and complexes.

Furthermore, rapidly advancing proteomic technologies will continue to provide detailed information of network molecular elements. Unknown network elements can be determined using well known techniques in the art and the possible characteristics, states and complexes can be inferred from the identity of the molecule for purposes of modeling of the network.

At block 106 of FIG. 3A, molecular entity graphs of identified network biomolecules are formulated that preferably include fixed attributes, variable attributes and a graph label. In one embodiment, a Molecular-entity Graph is a triple M=(V, E, A_{M}), where V is a set of labeled attributed vertices and E is a set of undirected edges. Vertices represent components. Vertex labels need not be unique; multiple vertices with the same label indicate components considered to be equivalent and may give rise to structural symmetry. Edges represent intra-molecular or intermolecular bonds between components. A molecular entity graph has a unique label and may have an optional set of attributes A_{M}. Molecular entity graphs reflect the level of abstraction in a model and largely define the model's scope.

Additional definition of the developed model comes from typing of the components and edges in molecular-entity graphs. Briefly, typing defines which attributes of a vertex are variable and which are fixed. Typing also defines the possible values of the variable attributes. Fixed attributes might include sequence, molecular weight, links to annotation sources, etc. Molecular weight is one example of a fixed attribute that might affect re-activity. An example of a variable attribute is phosphorylation status, which often affects binding activity.

Complex Graphs are formulated at block 108 of FIG. 3A from the list of formulated molecular entity graphs. A Complex Graph M_{Σ} is a connected set of molecular-entity graphs. A complex graph may be associated with an alphanumeric label, if desired, and an optional set of attributes. For example, many potential molecular entities contain a receptor dimer, which can be represented as a complex graph. It is important to consider complexes, because complexes can be observed experimentally and are often of functional significance. An example is provided by the case of a receptor that becomes phosphorylated only when it is complexed with a second receptor of the same type. Complex graphs are connected at the level of molecular-entity graphs, but because the vertices of a molecular-entity graph need not be connected, a complex graph may be unconnected at the level of component vertices. If the analysis is restricted to the consideration of binary interactions (the default assumption), then each vertex of a complex graph is connected by at most one edge. The label of a complex graph may be either assigned or derived from stoichiometry and molecule labels.

At block 110 of FIG. 3A, chemical species graphs are determined. A chemical-species graph G is a molecular-entity or complex graph with any and all variable attributes taking specific values. As discussed above, the material building blocks of a biochemical network are its components, molecules, and complexes. Chemical species, one of the two kinds of elements in a chemical reaction network, are particular configurations of these building blocks with specific internal states.

At block 112 of FIG. 3A, Component level type graphs are formulated. The molecular-entity graphs of a system, and all derivative graphs of a system, may be typed over a component-level type graph, which defines the types of vertices and edges in the system. A Component-level Type Graph (CTG) of a biochemical system comprises a pair (CV, CE), where CV is a set of vertex (component) types, and CE is a set of edge (bond) types. Each type is preferably associated with a set of attributes, which may be variable or fixed. Values of fixed attributes are defined, and the allowable values of variable attributes are enumerated or otherwise indicated. Any graph G of a system comprised of or derived from the system's set of molecular entity graphs is typed over CTG via a mapping g: G→CTG.

Pattern graphs are formulated at block 114 of FIG. 3A. Pattern graphs are derived from molecular entity graphs or complex graphs and can be considered to be sub-graphs of chemical species graphs. A Pattern Graph P=(V_{P}, E_{P}) is a set of molecular-entity and/or complex graphs. These graphs need not be connected. The components, molecular entities, and complexes of P may each be associated with a set of variable attributes. In addition, connectivity of the graphs of P to external components may be specified via an interface. For example, the Interface of a Pattern Graph I_{P} can be a partition of V_{P} into three sets: V_{P}=V⁰_{P}␣, V¹_{P}␣ V⁰¹_{P}, where V⁰_{P} is a set of components that cannot be bound to components external to the pattern graph, V¹_{P} is a set of components that must be bound to components external to the pattern graph, and V⁰¹_{P} is a set of components that are free to be either bound or unbound to components external to the pattern graph.

At block 116, chemical species graphs are matched with substantially identical pattern graphs to create an ensemble of chemical species graphs. The set of chemical-species graphs matching a pattern graph is an ensemble, because these graphs represent chemical species that all have a common reactivity or all contribute to a common quantity (the value of an output function). An ensemble of chemical-species graphs Ω_{P} is a set of chemical species graphs each matched by an identical pattern graph P. In the embodiment shown, a chemical species graph C=(V,E) is matched by a pattern graph P=(V_{P}, E_{P}) when the following three conditions are met:

1. there exists a subgraph C′=(V′,E′)⊂C isomorphic to P via an isomorphism f: P→C′;

2. f is consistent with the interface Iₚ; and

3. f preserves attributes of components, molecular entities, and complexes, e.g., for a vertex vεV_{P} attributes of f(v)εV′ fall within the set of attributes defined for vεV_{P}.

It can be seen that the chemical-species graphs containing multiple subgraphs isomorphic to a pattern graph may be matched multiple times. For example, the simple string pattern AB matches BAB twice. In another embodiment, “context” attributes are associated with vertices of a pattern graph to restrict or otherwise control the number of matches, which affects parameterization of reactions.

Function evaluation rules are specified at block 118 of FIG. 3A and applied to the ensembles of chemical species graphs derived at block 116. The observed results of an experiment typically correspond to the properties of ensembles of chemical species. Therefore, the ability to determine such properties is desirable so that model predictions can be tested. This capability is obtained by specifying a function evaluation rule. A “function evaluation rule” is a pattern P and a function of attributes of chemical-species graphs belonging to Ω_{P}. This function is referred to as an output function.

A function evaluation rule is processed by first finding the chemical-species graphs matched by the pattern graph of the rule and then calculating the value of the rule's output function. An example of an output function is a weighted sum of concentrations. A rule associated with this type of function is useful, for example, for determining the total concentration of a protein X in a particular state of phosphorylation when the protein may be distributed among numerous chemical species, as is usually the case. Concentrations of chemical species are weighted by the number of X proteins in each species. As another example, the output function may be a graph that matches graphs representing molecules and molecular complexes that include a fluorophore label, such as fluorescein isothiocyanate, and the sum generated by the rule is proportional to the total fluorescence observable when the flourophore is excited by light.

Accordingly, the chemical species element of a biochemical network can be defined that accounts for molecular components, such as protein interaction domains and the potential states of these domains as well as rate constants and concentrations and output functions. The graphical representations of molecular characteristics and associations between molecules and states that can change due to chemical reactions between the components of the network may also change with the application of graph rewriting rules. Chemical reactions are a second important element of the analysis of the biochemical reaction network.

Turning now to FIG. 3B, the various chemical species graphs may be subject to reaction rules for each of the possible reactions that take place within the system. In the preferred embodiment, the chemical reaction ρ comprises a set of reactant chemical species graphs R_{ρ}, a set of product chemical species graphs P_{ρ}, and a rate law v_{ρ}. Product chemical species graphs are preferably obtained from reactant chemical species graphs via graph rewriting consistent with observed chemistry.

Graph rewriting consistent with chemistry in the case of a closed system means that P_{ρ} is obtained from R_{ρ} via composition of the following operations:

1. Addition or removal of intra- or inter-molecular edge(s),

2. change of values of variables attribute(s), and

3. replacement of a molecular entity or set of molecular entities with another molecular entity or set of molecular entities having the same components.

For an open system, two additional operations are allowed: synthesis and degradation of a set of molecular entities. Degradation of a molecule means that its corresponding molecular-entity graph is removed (to a sink external to the system being modeled) along with any and all bonds to which it is connected. Synthesis of a molecule means that a new molecular entity appears (from a source external to the system being modeled). Finally, a second class of operations includes transport between compartments if compartment location may be included as a variable attribute of molecular entities in a multi-compartment system.

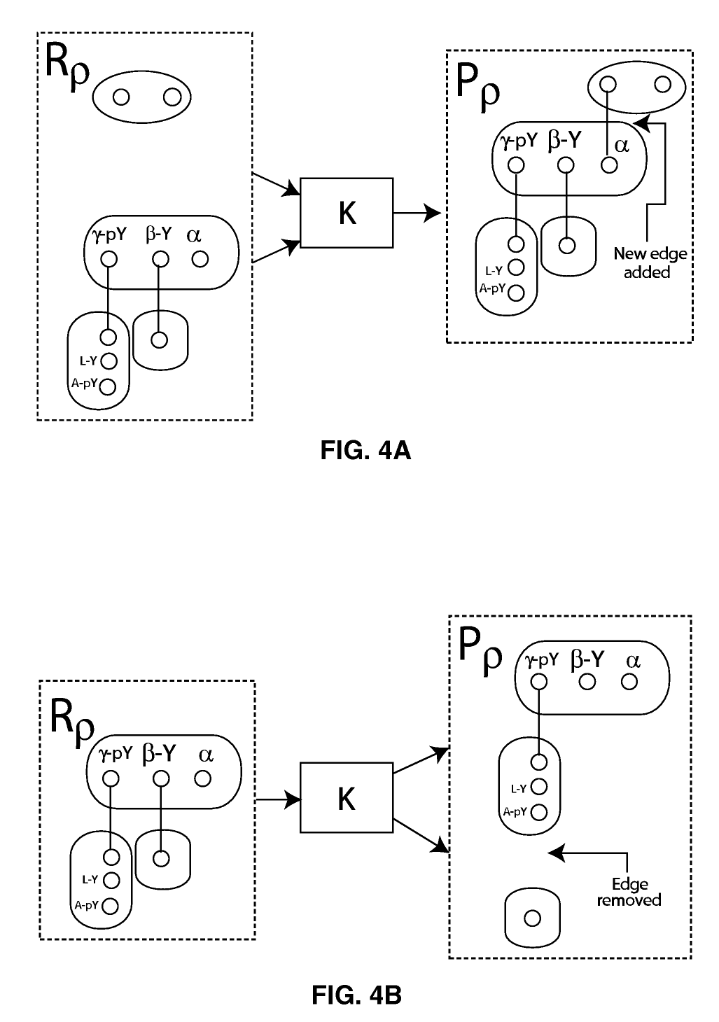

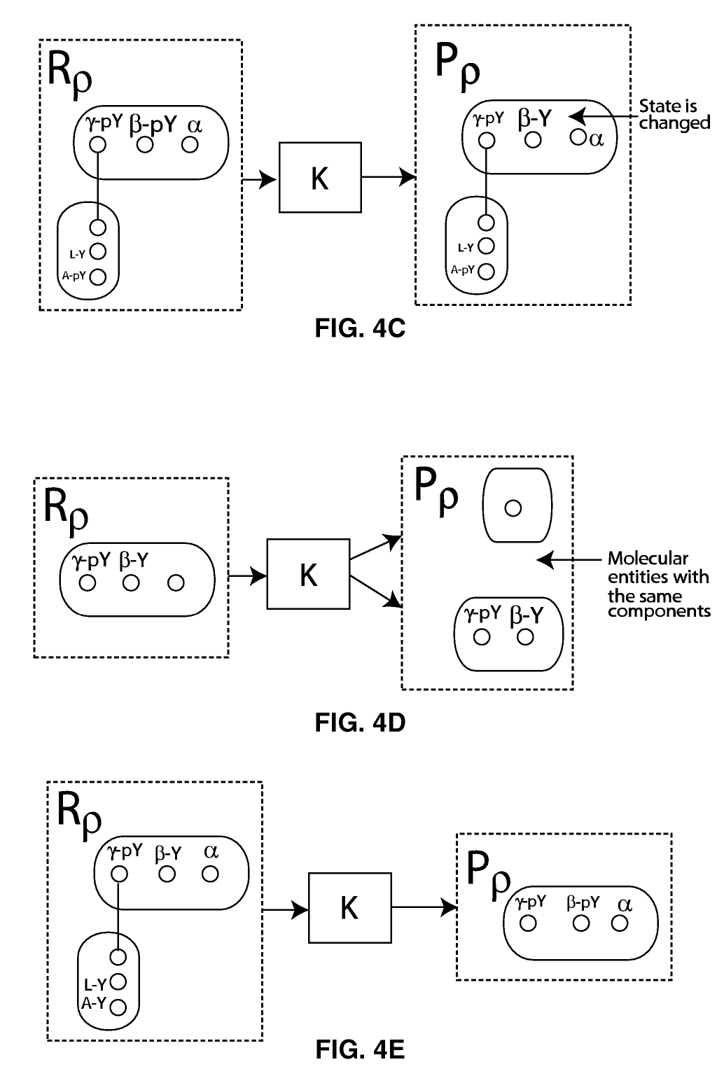

Referring also to FIG. 4A through FIG. 4E, different types of reaction and rewriting operations are shown. In FIG. 4A, the addition of an intermolecular chemical bond is illustrated. The breaking of an intermolecular chemical bond is shown in FIG. 4B. It will be seen that the breaking of a bond does not necessarily lead to two separate chemical species, because molecular entities may be connected by more than one bond and bonds may be intramolecular as well as intermolecular. FIG. 4C illustrates a change in an attribute value in a component. The replacement of a molecular entity with two molecular entities having the same components, so that one may model assembly and disassembly of a multimeric protein is shown in FIG. 4D or covalent reactions between proteins or proteolytic cleavage of a protein.

Finally, FIG. 4E illustrates the degradation of a molecular entity. Note that, as suggested by the layout of the diagrams in this figure, if the chemical-species graphs in R_{ρ} and P_{ρ} are each replaced with a single node, then a chemical reaction can be represented as a directed bipartite graph. The composition of the rewriting operations of a reaction implies a mapping f_{ρ} between vertices of R_{ρ} and P_{ρ}. This mapping must preserve, add, and remove molecular-entity graphs as units. In other words, if any vertex of a molecular entity in R_{ρ} maps to Ø, then all other vertices of this molecular entity must also map to Ø as seen in FIG. 4(e). Vice versa, for example, if some vertex vεM⊂P_{ρ} lacks a preimage, then no other vertices of M may have preimages. Furthermore, with the synthesis/degradation of molecular entities, f_{ρ} preserves components, i.e., vertices of chemical species in R_{ρ} and P_{ρ} are the same even if molecular entities are replaced with other molecular entities (FIG. 4D).

At block 120 of FIG. 3B, reaction rules are formulated for the potential reactions of the network chemical reactions. A reaction rule is a generalization of an individual reaction. It defines a class of chemical transformations from reactants to products. Reactants and products normally have common predictable properties. Reaction rules serve as generators of reactions, which can then be translated into mathematical or computational models. The generation of models through the automatic interpretation of reaction rules and the formalism of graphical reaction rules overcomes the limitations of writing models manually, which may be impossible. Graphical reaction rules also allow the connectivity of proteins in a complex to be explicitly and systematically represented. These rules expressively represent biomolecular interactions and the consequences of those interactions. The ability to incorporate the secondary structure of a complex in a model is needed when connectivity affects the reactivity of a complex.

At block 120 of FIG. 3B, a formulated reaction rule is preferably a graph transformation rule. In one embodiment, for example, the graph transformation rule r: RP→PP, a rate law v, an application condition α, and precedence index N, where:
1. A disjoint union of m reactant pattern graphs RP is used to match and select m reactant chemical species Cr.
2. The transformation rule r includes a component-level mapping function f: RP→PP consistent with chemistry. It maps RP to a set of n product pattern graphs PP. A set of reactant chemical species Cᵣ undergoes transformation by replacing the image of RP in Cᵣ with PP via mapping function f. Dangling edges are preferably removed. This process of graph rewriting corresponds to the well-known single-pushout approach. To avoid ambiguity while embedding PP in Cᵣ, any vertex of RP in V_{RP}⁰ of the interface I_{RP} must remain in the same set in PP, i.e. f(V_{RP}⁰)⊂V_{PP}⁰, in one embodiment.
3. The rate law v is a function of rate parameters, such as a single-site rate constant, and properties of chemical species Cᵣ, such as their concentrations.
4. The application condition α may include, for example, a pattern selecting species that may not serve as reactants.
5. The Precedence Index N is preferably the priority of reactions that are generated by the rule. In addition, it is sometimes convenient to specify a rule that will generate reactions that replace a subset of reactions generated by another rule.

In one embodiment, a negative application condition can be specified by assigning a zero-valued rate law to a rule. All reactions with lower precedence generated by other rules are overridden. For example, an inhibitor of an enzyme can be introduced to a model. In this case, an old rule that generates reactions catalyzed by the enzyme can be overridden by a new rule that additionally contains the inhibitor in RP and generates with higher precedence a reaction with a zero-valued or reduced rate.

A seed set of chemical species graphs are selected at block 122 of FIG. 3B. Seed chemical species graphs are initially selected that represent at least one of each of the molecules within the network. The seed species are selected by the modeler, and the choice of seed species can affect the biochemical reaction network generated by application of rules. Thus, the set of seed species is an integral part of a model specification. A preferred method for specifying the seed species is to include all species present in a system before a peturbation of the system, such as the introduction of a signal molecule. However, other sets of seed species may yield the same result, as would become apparent to one who practices the invention.

At blocks 124, 126 and 128 of FIG. 3B, a biochemical reaction network can be generated through iterative application of a set of reaction rules to a seed set of chemical species until no further change is possible (exhaustive generation) or a specified termination condition is reached (such as iteration until generation of a given number of product species or reactions).

The process of applying reaction rules to a set of distinct chemical species graphs C⁰ at block 124 and block 125 preferably consists of the following steps. For each chemical species C matched by RP, a transformation replaces RP in C with PP according to a procedure of graph rewriting, which corresponds to the standard single-pushout approach. In this embodiment, the application of reaction rules to a set of selected chemical species graphs C⁰ is conducted according to the following rules:
1. For each reaction rule r_{m,n}, RP₁+ . . . RPₘ→PP₁+ . . . PPₙ, identify all sets of species graphs in C⁰ that qualify as reactants. Then, for each RPᵢ, find all matching species graphs CᵢεC⁰.
2. For each set of reactant species ␣Cᵢ, define a chemical reaction (graph transformation) by replacing the image of ␣RPᵢ in ␣Cᵢ with ␣PPⱼ, In this operation, attributes of vertices in ␣Cᵢ that do not differ between the corresponding vertices of ␣RPᵢ and ␣PPⱼ are preserved. Incident edges of ␣Cᵢ not indicated in ␣RPᵢ or ␣PPⱼ are also preserved. Any edge (l,c) between a vertex lεRPⱼ and cεC\U ␣RPᵢ is either replaced with an edge (f (l), c), if f(l)ε␣PPⱼ or removed, if f(l)=Ø. Assign the precedence index N of the reaction rule to each reaction.
3. Applying all reaction rules to the set of seed species, generate a list of distinct reactions R⁰. If the list R⁰ contains identical reactions with different precedence indices, delete reactions with indices less than the maximum index. All identical reactions of the same precedence remain in R⁰.
4. Identify chemical species that are products in the list R⁰ but that are not isomorphic to any in the list C⁰ to obtain a list of new species graphs C¹.

After the initial reaction rules are applied, the network generation procedure is continued by iteratively applying each of the reaction rules to the set of species in ∪ᵢ₌₀^{k}Cⁱ, where k is a counter that is updated after each round of rule application. Note that reactions need only be generated when reactant species include at least one reactant in the list C^{k}. After each round of exhaustive application of the rules, we obtain a list of new reactions R^{k} and a list of new product species C^{k+1}. Termination occurs when either no new species are found or a specified termination condition is satisfied.

Rule evaluation can be terminated by specifying an arbitrary cutoff for chain size, number of species, etc. or a maximum number of iterations of rule evaluation. Care must be taken with this approach to ensure that a generated network is of sufficient size to encompass the species populated in a simulation. Alternatively, rule evaluation can be embedded in the network simulation. With this approach, network elements (species and reactions) are generated as needed and arbitrary termination of network generation is avoided. The fact that a set of reaction rules can generate sets of species and reactions of unbounded size demonstrates that membership of a given species in a reaction network is semi-decidable, meaning that membership cannot generally be ruled out in a finite number of steps. Also, in general, it cannot be determined if evaluation of a set of rules will eventually terminate in the absence of a specified termination condition, such as a maximum number of iterations.

The iterative application of reaction rules can be illustrated with a single rule and a set of two seed species. For this illustration, the two seed species are a free ligand with two identical binding sites and a free receptor with two identical binding sites. The rule, which is a graph rewriting rule, defines a class of irreversible biomolecular association reactions in which the products are polymer-like chains of ligands and receptors. In this illustration, the rule indicates that a bond can form between a ligand with a free binding site and a receptor with a free binding site, if the ligand and receptor are not already associated directly or indirectly.

The first round of rule evaluation has a first reaction that produces a 1:1 ligand-receptor aggregate, a new species. The next round of rule evaluation generates two new products, which are two new species 1:2 and 2:1 receptor aggregates. Each round of rule evaluation generates at least one new reaction in which a product is a new ligand-receptor aggregate composed of more molecules that any aggregate previously generated.

Accordingly, the protein-protein interactions are specified as generators of chemical reactions or reaction events and species. A rule can be viewed as a definition of a reaction class. The relevant features of an interaction can often be identified because of the modularity of protein catalytic and interaction domains.

Graphical reaction rules have precise interpretations that can be visualized and can replace diagrammatic interaction maps currently in use. A set of well-posed rules unambiguously specifies a reaction network, and a model for this network can be generated through a computational procedure that interprets the rules. Because the procedure is automatic, once rules are specified, very little mathematical or computational expertise is required in principle to obtain a mathematical model. The ability to generate models through automatic interpretation of rules overcomes limitations of writing models manually, which may be impossible.

Graphical reaction rules are also close in form to the type of biological knowledge usually available about a system, which may consist mainly of a list of proteins, their functional components, and their binding and catalytic activities, even for a well-studied system. Such rules allow the connectivity of proteins in complexes to be explicitly represented and tracked in a model. Finally, rules for biomolecular interactions may be useful for high-throughput modeling of large numbers of systems and for development of models that include a large number of distinct interacting biomolecules. Rules are independent units of a model specification and sets of rules are compositional, which allows models to be built incrementally. The independence of rules facilitates not only incremental model building but also the consideration of alternative models and mechanistic hypotheses. For example, to introduce a protein-protein interaction in a system to investigate its effect, one can simply add an appropriate rule instead of adding and modifying possibly large numbers of interrelated equations or lines of code. If rules are stored in a machine-readable format in an electronic database, they can be reused. Rules can be assembled in different ways to define different systems, which may share some components, and models for different parts of a larger system can be integrated by combining the corresponding sets of rules. In principle, crude models of a large size could be built at present from information of pairwise protein-protein interactions currently catalogued in electronic databases. With standardization and time, large numbers of graphs and graph rewriting rules in machine readable form can be archived in an electronic database or a federation of databases that facilitates the generation of reaction networks. For example, graphs that represent drug compounds and their target molecules encoded in machine readable format can be used to investigate the effects of drug therapies.

At block 130, optional simulations that predict the dynamics of the elements of the network can be conducted. For example, the network generated by the methods can be used in a simulation procedure that predicts the dynamics of the network, as in conversion of the network into a coupled system of ordinary differential equations and numerical integration of these equations using well-known techniques of the art.

For example, the methods can be used to model the kinetics of phosphorylation of individual tyrosine residues of proteins when the phosphorylation events are triggered by the binding of a ligand to a cell-surface receptor. Likewise, the methods can be used to model the interactions of a protein transcription factor with its binding partners, including protein co-factors, sites on DNA, the transcriptional machinery, and small-molecule metabolite effectors. There is a wide variety of potential applications.

The invention may be better understood with reference to the accompanying examples, which are intended for purposes of illustration only and should not be construed as in any sense limiting the scope of the present invention as defined in the claims appended hereto.

### EXAMPLE 1

The graphical rule based modeling of a biochemical network may be illustrated by FcεRI mediated signal transduction. FcεRI is a high affinity receptor for immunioglobin E (IgE) and is the Fc receptor found on granulocytes involved in allergic reactions and defense against infections. Cross-linking of at least two IgE molecules and their Fc receptors on the surface of a granulocyte will trigger the rapid release of various molecules from its granules.

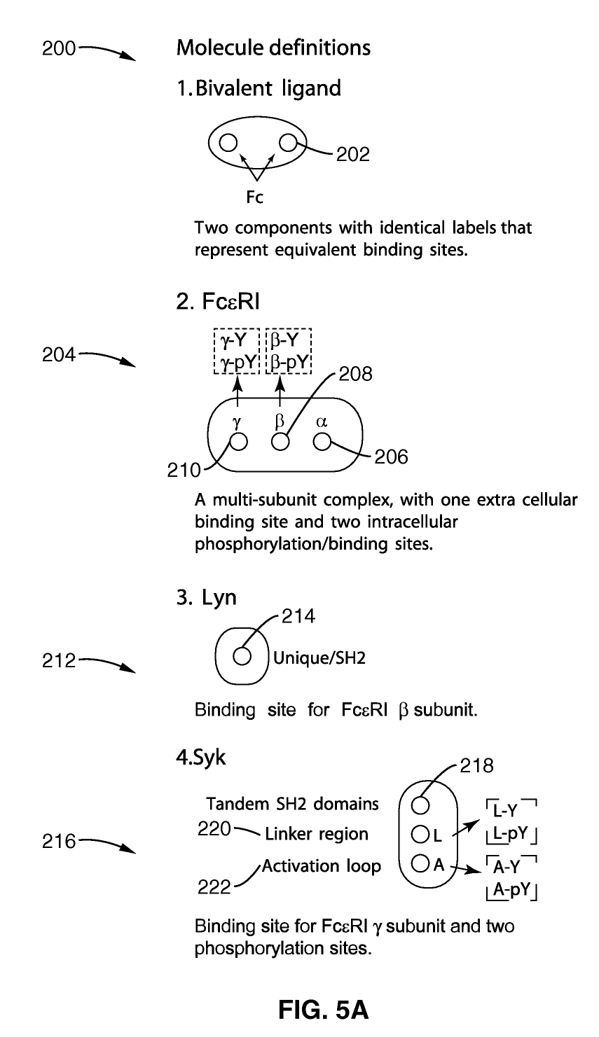

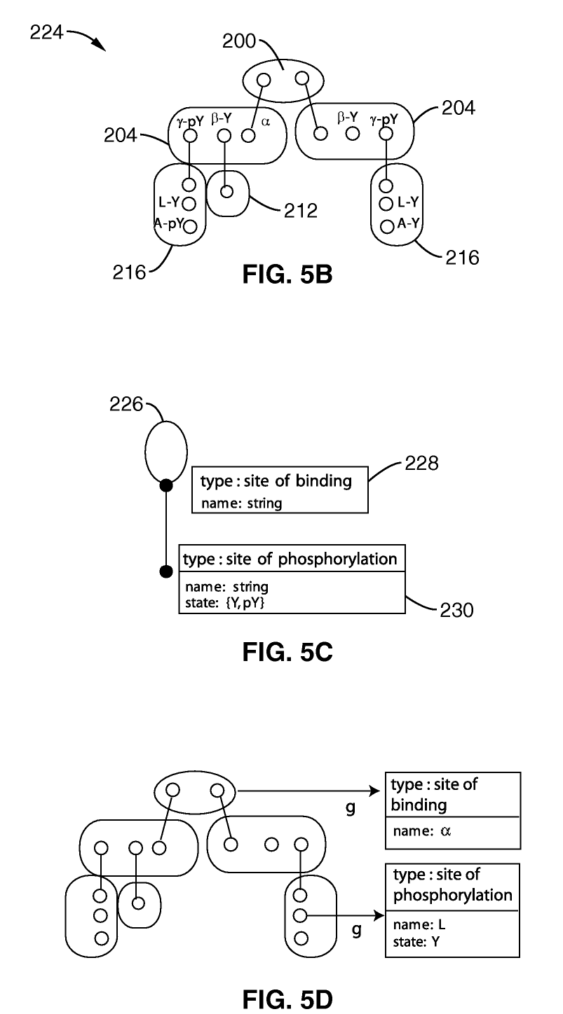

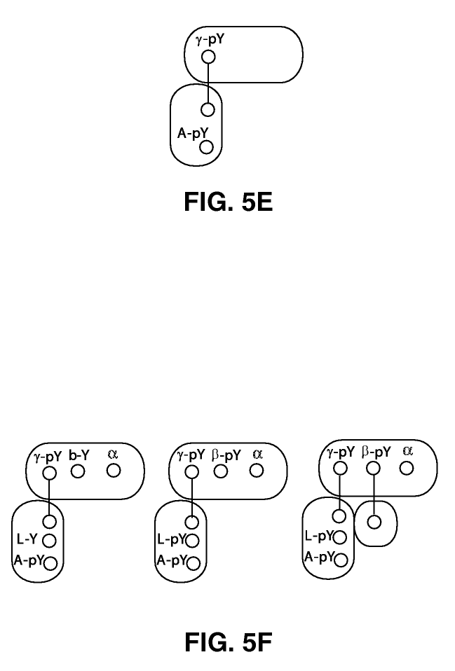

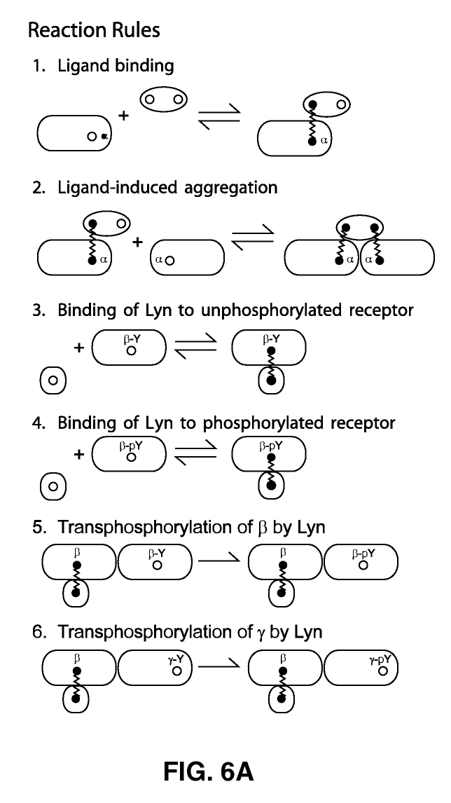

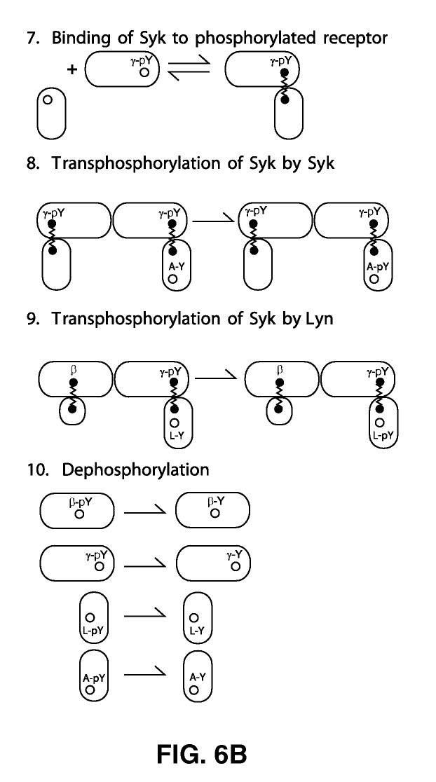

Early signaling events mediated by the immune recognition receptor FcεRI, are shown through consideration of the interactions of a ligand and three signaling proteins including two protein kinases Lyn and Syk and FcεRI. These four molecules are represented graphically in FIG. 5A. When these four molecules are subject to the ten reaction rules of FIG. 6A and FIG. 6B, the method of the invention generates 354 distinct chemical species that are connected through 3680 unidirectional reactions.

Referring to FIG. 5A, molecular entity graphs for the four proteins are shown. Note that edges are not included, even though the components of the molecules are physically connected. The bivalent ligand 200 is comprised of two identical binding domain (Fc) nodes 202 that represent equivalent binding sites.

The multi-subunit complex of the FcεRI receptor 204 consists of three components 206, 208 and 210 representing the α, β and dimeric γ chains of the receptor respectively. The complex has one extracellular binding site α that binds the ligand and two intracellular phosphorylation/binding sites. The β and γ components have phosphorylation state attributes like A and L below.

The protein tyrosine kinase “Lyn” 212 includes a single component that includes the unique and Src homology 2 (SH2) domains 214 of this protein. This is the binding site for the β subunit of FcεRI.

Vertices within the protein tyrosine kinase “Syk” 216 represent three components: 1) tandem SH2 phosphotyrosine binding domains 218, 2) a linker region (L) 220 and 3) an activation loop (A) 222. Components L and A have a ‘state’ attribute that can take two values: Y and pY, corresponding to “not phosphorylated” and “phosphorylated” respectively.

The complexity of the underlying biochemical network can be seen in the possible combinations of the proteins. For example, there are four states of the β subunit of FcεRI that are possible, namely, unphosphorylated and unbound, unphosphorylated and bound to Lyn, phosphorylated and unbound and phosphorylated and bound to Lyn. There are six states that are possible for the γ subunit. Accordingly, there are 24 possible modification states for the cytosolic portion of the FcεRI receptor.

Chemical species graphs are identified and can be either a single molecule having all of its components fully defined or a set of connected molecules with each molecule in the set having all of its components fully defined. FIG. 5B is an example of a chemical species graph 224. In the example shown, the two available binding sites of the ligand 200 are bound to the α subunits of two FcεRI molecules. The γ subunits of the FcεRI molecules are bound to the SH2 domains of Syk and one β subunit of one FcεRI is bound to Lyn while the β subunit of the second FcεRI remains unbound.

The molecular-entity graphs of a system, and all derivative graphs of a system, are typed over a component-level type graph, which defines the types of vertices and edges in the system. A component-level type graph (CTG) is illustrated in FIG. 5C. As shown in FIG. 5C, the components of the molecules in the FcεRI model are preferably considered to belong to one of two types, namely, each component 226 is a site of binding 228 and/or a site of phosphorylation 230. A site of phosphorylation has a variable attribute, which has two possible values, Y (not phosphorylated) or pY (phosphorylated). Furthermore, components α, β, γ, Fc, unique/SH2, and SH2 are sites of binding. Components β, γ, L, and A are sites of phosphorylation. The type graph shown in FIG. 5C further illustrates that two types of bonds are considered. A bond is allowed between two binding sites or between a binding site and a phosphorylation site.

A typing mapping is partially illustrated in FIG. 5D. In this example, the chemical species of FIG. 5B is typed over the Component-Level Type Graph (CTG) of FIG. 5C by the typing mapping g.

A pattern graph is illustrated in FIG. 5E. A pattern graph is a set of molecular-entity graphs and/or complex graphs. In the example of FIG. 5E, the pattern graph may be specified by the symbol used as a node (open, half-filled or filled circle).

A pattern graph is used to define an ensemble of chemical species graphs. They appear in reaction rules and function evaluation rules, described previously, and they can be considered subgraphs of chemical-species graphs. A set of chemical-species graphs matching a pattern graph is referred to as an ensemble, because these graphs represent chemical species that all have a common reactivity or all contribute to a common quantity (the value of an output function).

An example of a graph from an ensemble of chemical species graphs matched by the pattern graph of FIG. 5E is shown in FIG. 5F. Note that chemical-species graphs containing multiple subgraphs isomorphic to a pattern graph may be matched multiple times.

Turning now to FIG. 6A and FIG. 6B, reaction rules for the FcεRI model are shown. The reaction rules shown in the example of FIG. 6 include: ligand binding, ligand-induced aggregation, binding of Lyn to an unphosphorylated receptor, binding of Lyn to a phosphorylated receptor, transphosphorylatidon of β by Lyn, transphosphorylation of γ by Lyn, binding of Syk to phosphorylated receptor, transphosphorylation of Syk by Syk, transphosphorylation of Syk by Lyn and dephosphorylation. Other types of reactions may include the addition of an intermolecular chemical bond and the breaking of an intermolecular chemical bond. Note that breaking a bond does not necessarily lead to two separate chemical species, because molecular entities may be connected by more than one bond and bonds may be intramolecular as well as intermolecular. Reaction rules may also include the change of a component's attribute value, the replacement of a molecular entity with two molecular entities having the same components or the degradation of a molecular entity.

The generation of a chemical reaction network normally begins with the designation of an initial set of chemical species and a set of reaction rules. A typical starting point for network generation would be the set of individual molecules with each component in its resting internal state. A set of chemical species in the FcεRI case may include: free ligand, free Lyn, free FcεRI, the Lyn-FcεRI complex and free Syk. The iterative application of the reaction rules shown in FIG. 6 to this initial set of chemical species will generate a reaction network containing 354 chemical species and 3680 reactions.

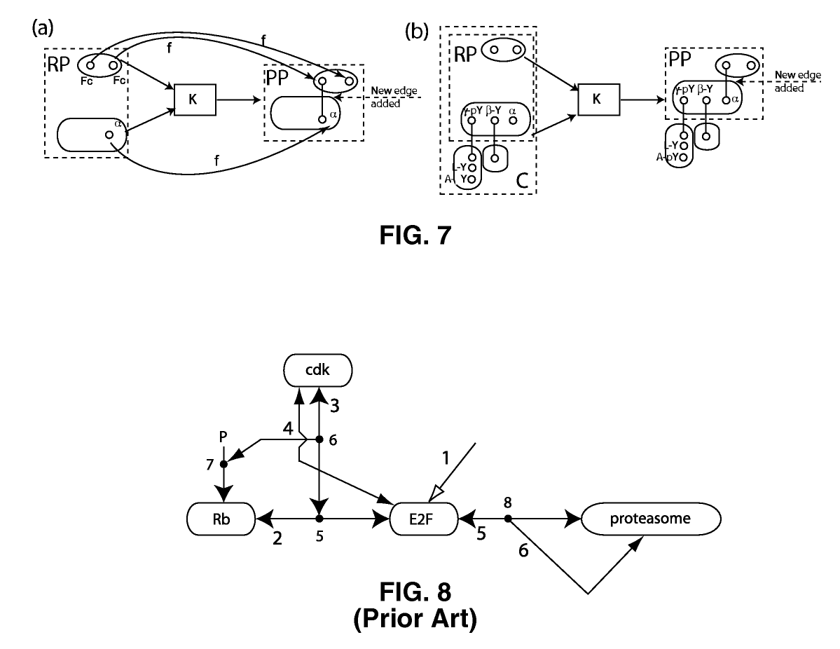

FIG. 7(a) is an example of a reaction rule for ligand receptor binding. It can be seen that the rule consists of a reactant pattern graph RP, a product pattern graph PP and a mapping f illustrated with arrows. In the example shown in FIG. 7(a), the interface of RP specifies that two Fc components and an a component of RP should be unbound. The rule generates a reaction in which one Fc component is bound to the α component while the other Fc component is unaffected. The remaining components of species matched by the rule are also unchanged. An example of a reaction that may be generated by the application of the reaction rule of FIG. 7(a) is shown in FIG. 7(b). When a reaction rule is introduced to a set of chemical species, a class of reactions is defined. Models can be generated through the automatic interpretation of rules and the connectivity of components can be tracked systematically and explicitly. One can generate a system of coupled ordinary differential equations, for example, or a stochiastic simulation algorithm (a Monte Carlo method for simulating discrete event reaction kinetics).

### EXAMPLE 2

A second illustration of the use of graphical reaction rules to represent protein-protein interactions and their consequences in comparison with a conventional diagram is shown in FIG. 8 and FIG. 9. Two representations of a model for the phosphorylation of the retinoblastoma protein (Rb) by a cyclin-dependent kinase are generally shown in FIG. 8 and FIG. 9. The first representation is shown in FIG. 8 and is a diagram of molecular interactions and drawn according to the scheme proposed by Kohn, Molecular Interaction Maps As Information Organizers And Simulation Guides, Chaos 11: 84-97, (2001). One disadvantage of this diagrammatic approach is the need to represent each binary complex as a separate numbered dot since the diagram only illustrates individual species and reactions. This can become problematic when the number of reactions and complexes is large.

In contrast, in the graphical rule-based approach, interactions are specified in the form of rules and the complexes implied by these rules can then be identified in an automatic procedure. The number of reaction rules needed to represent a system is related to the number of components of the system, which in general is far less that the number of possible chemical species and reactions.

FIG. 9 depicts the phosphorylation of the retinoblastoma protein (Rb) by a cyclin-dependent kinase (cdk). The process begins when Rb forms a complex with E2F to create the Rb-E2F complex. This complex can be bound by cdk to create a complex Rb-E2F-cdk, which can then dissociate into E2F, cdk, and the phosphorylated form of Rb. The concentration of E2F is determined by its rate of synthesis and its rate of degradation in the proteasome. The degradation process includes the binding of E2F to the proteasome and the creation of an E2F-proteasome complex leading to the disappearance of E2F from the complex.

It can be seen that network models can be derived from a set of graph rewriting rules that are associated with rate laws and other parameters applied to a set of seed chemical species graphs. Reaction rules are expressive enough to convey information about the contextual constraints on a protein-protein interaction and the parts of the proteins responsible or affected by an interaction. Such models can also include all of the experimental information available about the components and interactions of the biological network.

Once a network has been generated it can serve as a basis for stochastic and other simulations. Because chemical reaction networks derived from rules may be large, one embodiment confirms the model for consistency with known experimental evidence.

Rules can be processed to automatically generate a mathematical or computational model for a system that enables explanatory and predictive insights into the dynamics of the system. Graphical rules are independent units of model specification that facilitates easy model revision. Conventional models require the change of a large number of lines of code or equations to modify a protein-protein interaction. A protein interaction can be introduced or modified in the present invention simply by adding or changing a single rule that represents the interaction of interest. Graphs and graphical rules can also be easily encoded in a machine-readable format to enable electronic storage and exchange. The formalism supports the generation of a list of reactions in a system, which can be used to derive different types of physiochemical models that can be simulated and analyzed in different ways.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A method for generating a biochemical reaction network, comprising:
identifying the components and states of molecular entities within a system;
generating molecular entity graphs representing the components and states of said molecular entities;
formulating graph transformation rules representing the potential reactions and products of said molecular entities; and
applying said graph transformation rules iteratively to said molecular entity graphs until no new graphs are generated or a termination condition is satisfied to provide a network of biochemical reactions.

2. A method as recited in claim 1, wherein said molecular entity comprises a plurality of molecular entities.

3. A method as recited in claim 1, wherein said molecular entity graphs comprise nodes representing components of said molecular entities and edges representing associations between one of said components with another component.

4. A method as recited in claim 3, wherein said molecular entity graphs further comprise edge labels.

5. A method as recited in claim 3, wherein said nodes representing said components of said molecular entity graphs comprise variable and fixed attributes.

6. A method as recited in claim 5, wherein said fixed attributes of said nodes of said molecular entity graphs are attributes selected from the group of attributes consisting essentially of molecular weight, sequence and annotation sources.

7. A method as recited in claim 6, wherein said variable attribute of said nodes comprises phosphorylation status.

8. A method as recited in claim 1, wherein said graph transformation rules comprise graph rewriting rules.

9. A method for generating a model of complex biochemical networks, comprising:
specifying an initial set of molecular entities within a system;
identifying possible physical and chemical states of molecules, components and complexes of said molecular entities to provide a set of chemical species;
representing graphically said molecules, components, complexes and states of said chemical species;
specifying graph transformation rules for classes of products and reactions of said chemical species;
applying said graph transformation rules on an initial set of graphs of said chemical species to produce a first generation set of graphs; and
applying iteratively said graph transformation rules on said first generation set and upon on all subsequent generations of sets of graphs produced by the subsequent application of said graph transformation rules on the subsequent generations of sets of graphs to produce a model.

10. A method as recited in claim 9, further comprising an output function associated with a group of chemical species.

11. A method as recited in claim 9, wherein said graph transformation rules comprise graph rewriting rules.

12. A method as recited in claim 11, wherein said graph rewriting rules comprise:
the addition and removal of intra-molecular edges;
the addition and removal of inter-molecular edges, the change of values of variable attributes; and
the replacement of a molecular entity or set of molecular entities with another molecular entity or set of molecular entities having the same components.

13. A method as recited in claim 9, wherein said graph transformation rules comprise reaction rules of group graphs representing reactants, group graphs representing products and a rate law.

14. A method as recited in claim 13, further comprising pattern graphs where components, molecular entities and molecular complexes are associated with variable attributes of said molecular entities.

15. A method as recited in claim 14, wherein said graph transformation rules further comprise function evaluation rules.

16. A method as recited in claim 15, wherein said function evaluation rule comprises:
matching said chemical-species graphs with said pattern graphs; and
calculating a value of the output function of said function evaluation rule.

17. A method as recited in claim 9, wherein said initial set of graphs comprises chemical species graphs matched with identical pattern graphs.

18. A method as recited in claim 9, wherein the application of said graph transformation rules comprises:
applying a reaction rule to said initial set of chemical species to identify a reactant group of chemical species corresponding to each reactant group graph;
replacing subgraphs of each reactant species matching the reactant group graphs with the corresponding product group graphs to define a group of product species; and
checking a current list of chemical species and adding members of said group of product species to the list that are not already present.

19. A method as recited in claim 9, further comprising:
associating a mathematical function with a group of chemical species.

20. A method as recited in claim 9, further comprising:
archiving graphs and graph transformation rules in a database in computer readable form.

21. A computer system for modeling biochemical networks, comprising:
a computation device configured for receiving data input;
a software program to operate said computation device, said program performing the operations of:
graphically representing molecules, components and chemical states of an initial set of molecular entities within a system;
applying specified graph transformation rules for classes of products and reactants of molecular entities within said system upon a seed set of graphs to produce a first generation set of graphs;
iteratively applying graph transformation rules to subsequent generations of graphs until a termination condition occurs; and
forming a model of molecular entities within said system; and
a user interface allowing the user to view computed model results and provide graphical and text input to said computation device.

22. A computer system for modeling biochemical networks as recited in claim 21, wherein said data input to said computation device comprises graphs and graph transformation rules obtained from a database of graphs and graph transformation rules.

23. A computer system for modeling biochemical networks as recited in claim 21, wherein said software is configured to simulate network activity.
